(19) United States
(12) Patent Application Publication
(10) Pub. No.: US 2015/0250922 A1
(43) Pub. Date: Sep. 10, 2015

(54) POLYMERIC HYDROGEL FOR ACCELERATING WOUND HEALING

(71) Applicants: Megan Cole, Alpharetta, GA (US); John Cole, Alpharetta, GA (US)

(72) Inventors: Megan Cole, Alpharetta, GA (US); John Cole, Alpharetta, GA (US)

(21) Appl. No.: 14/201,573

(22) Filed: Mar. 7, 2014

(51) Int. Cl. A61L 27/18; A61L 27/54; A61N 5/06; A61M 5/19; A61M 5/28; A61L 27/52; A61M 37/00

(52) U.S. Cl. CPC A61L 27/18; A61L 27/52; A61L 27/54; A61M 37/00; A61M 5/19; A61M 5/284; A61N 5/062; A61N 5/0617; A61L 2300/236

(57) ABSTRACT

A method for creating a polymeric hydrogel for accelerating wound healing. The method includes applying at least one monomer solution to a tissue area of a patient. The monomer solution includes a monomer and a therapeutic agent. The method also includes initiating a polymerization process in the applied monomer solution to create a cross-linked network to deliver the therapeutic agent to the tissue area.

### CROSS-REFERENCE TO RELATED APPLICATIONS

Not applicable.

### BACKGROUND OF THE INVENTION

Male pattern baldness, or androgenic alopecia, may occur when one or more hair follicles begin to shrink. Small hair follicles may give rise to shorter, finer hairs. Eventually a very small follicle may be left with no hair inside at all. Male pattern baldness usually proceeds in a familiar pattern: it begins on the crown of the head and/or with a receding frontal hairline, and then progresses rearward.

In hair transplantation procedures, hair follicles may be extracted from a patient's scalp, referred to as a “donor” region, and may then be implanted into another area, referred to as a “recipient” region. However, after performing a hair transplantation procedure the physical appearance of the donor region may be adversely affected if, for example, a substantial number of hair follicles are removed from the donor region.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a method for creating a polymeric hydrogel for accelerating wound healing. The method includes applying at least one monomer solution to a tissue area of a patient. The monomer solution includes a monomer and a therapeutic agent. The method also includes initiating a polymerization process in the applied monomer solution to create a cross-linked network to deliver the therapeutic agent to the tissue area.

Another example embodiment includes a system for creating a polymeric hydrogel for accelerating wound healing. The system includes a first apparatus configured to apply a monomer solution to a tissue area of a patient. The monomer solution includes a monomer and a therapeutic agent. The system also includes a second apparatus configured to initiate a polymerization process in the applied monomer solution to create a cross-linked network to deliver the therapeutic agent to the tissue area.

Another example embodiment includes a system for creating a polymeric hydrogel for accelerating wound healing. The system includes a double barreled syringe configured to apply a monomer solution to a tissue area of a patient. The monomer solution includes a first monomer in a first barrel of the double barreled syringe, a second monomer in a second barrel of the double barreled syringe, and a therapeutic agent in the first barrel. The system also includes an apparatus configured to initiate a polymerization process in the applied monomer solution to create a cross-linked network to deliver the therapeutic agent to the tissue area.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of a portion of a hair growing tissue;

FIG. 2 illustrates an example of a hair growing tissue in detail;

FIG. 3 illustrates an example of a representative multi-arm PEG-thiol monomer;

FIG. 4 illustrates an example of an acrylate monomer;

FIG. 5 illustrates an example of a cross-linked network;

FIG. 6 illustrates an example of a syringe for providing one or more solutions containing one or more monomers to a patient's scalp or skin tissue;

FIG. 7 illustrates an example of an area of a patent's hair growing tissue having a wound;

FIG. 8 illustrates an example of an area of a patent's hair growing tissue having a wound with monomer solutions applied;

FIG. 9 illustrates an area of a patent's hair growing tissue having a wound with a cross-linked network disposed on the wound; and

FIG. 10 illustrates a process 1000 for generating a cross-linked network.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

In a hair transplantation procedure, hair follicles may be extracted from a patient's scalp, referred to as a “donor” region, and may then be implanted into another area, referred to as a “recipient” region. Such a process may be referred to as “follicular unit extraction.” However, after performing a hair transplantation procedure the physical appearance of a donor region may be adversely affected if a substantial number of hair follicles have been removed from the donor region. I.e., by removing a group of hair follicles from a donor region, the donor region may thereafter be deficient in a number of hair follicles.

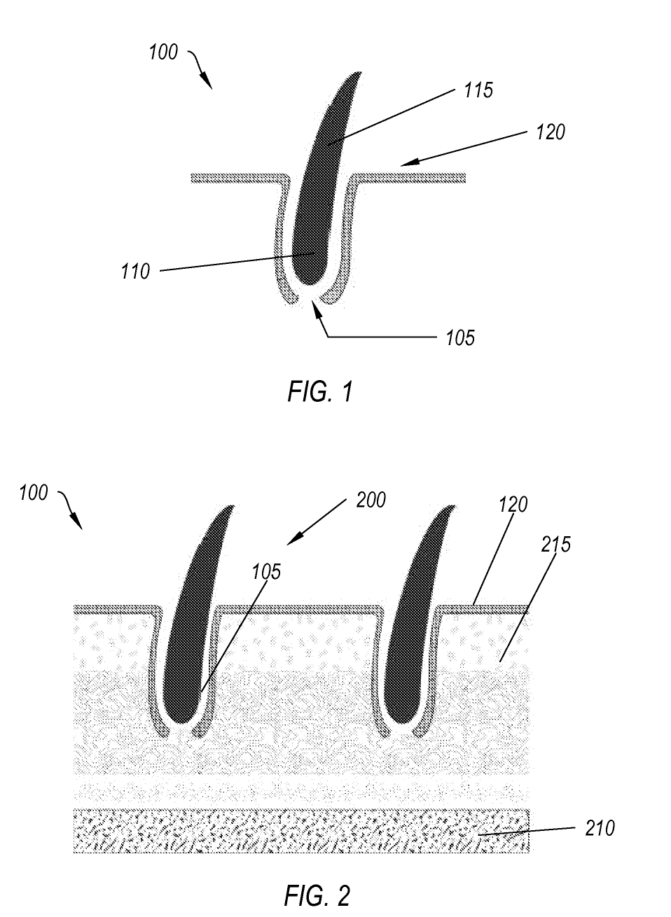

FIG. 1 illustrates an example of a portion of a hair growing tissue 100. For example, the hair growing tissue 100 can include a scalp or other area of a patient's skin. The hair growing tissue 100 may include a hair follicle 105. Hair follicle 105 may include a root 110 and a hair shaft 115. Hair shaft 115 may also be referred to as a “hair stem.” Root 110 may be located below a surface 120 of hair growing tissue 100, and hair shaft 115 may extend from root 110 to a location beyond or above surface 120. During a hair transplant procedure a hair follicle 105 (or group of hair follicles 105) may be surgically removed from a donor region and implanted into a recipient region. E.g., a grouping of two or more adjacently located hair follicles may be removed from a donor area and located to a single recipient region or multiple recipient regions.

FIG. 2 illustrates an example of a hair growing tissue 100 in detail. The hair growing tissue 100 may include several layers of skin. In particular, hair growing tissue 100 may include a surface 120 comprising an epidermis, dermis, and subcutis (subcutaneous) layer. Hair growing tissue 100 may include one or more additional layers disposed below surface 120. For example, galea aponeurotica 205 may be disposed below surface 120. A cranial bone 210 may be disposed below the subcutaneous tissue 215. It should be appreciated that one or more layers of connective tissue, mater, or fluid filled space may be disposed between the subcutaneous tissue 215 and the cranial bone 210. Additionally, one or more hair follicles 105 may be disposed within a hair growing tissue 100 between surface 120 and the subcutaneous tissue 215.

A hair growing tissue 100 may include various stem cells capable of regenerating one or more hair follicles 105. For example, stem cells may be located in a layer of skin below hair follicles 105 (as shown in FIG. 2). Stem cells may be prompted to grow one or more hair follicles 105 if an extracellular matrix is formed or applied to an area of hair growing tissue 100 in the vicinity of one or more follicular extraction sites during a hair follicle 105 harvesting operation. For example, if an incision of approximately 2.0 mm, a minimal depth considering the thickness of the patient's scalp, is made to remove a hair follicle 105 additional layers of skin beneath the extraction point may remain unaffected by the incision. One of skill in the art will appreciate that a hair follicle 105 may project out of the scalp at an angle and require the incision to be made at a similar angle to extract the hair follicle 105. The minimal depth incision provides a follicular extraction mechanism wherein approximately 2.0 mm or more of skin persists and stem cells capable of regenerating hair follicles 105 may be contained within unharmed areas of the scalp. As used in the specification and the claims, the term approximately shall mean that the value is within 10% of the stated value, unless otherwise specified.

Application of an extracellular matrix to a patient's scalp may be capable of promoting regeneration of hair follicles 105 that have previously been surgically extracted during a follicle harvesting procedure at a rate of approximately 10-60%. As used herein, “extracellular matrix” may refer to a part of human or animal tissue that provides structural support to cells. Extracellular matrix cells may regenerate and heal tissues, including skin tissue and may prevent the immune system from triggering an injury response, which may lead to inflammation and scar tissue formation, while facilitating surrounding cells to repair tissue.

An extracellular matrix may play an integral role in connective tissue. In particular, the extracellular matrix may provide support for cells and may have an ability to segregate tissues from one another and regulate intercellular communication. A formation of extracellular matrix may aid growth, wound healing, and fibrosis.

An extracellular matrix can include collagens, elastins, or other structural proteins. Collagen is a protein source of an extracellular matrix and may also comprise a majority of bone matrix content. Collagen may be present in fibular proteins of an extracellular matrix to provide structural support to resident cells. Moreover, the extracellular matrix can include fibrous proteins or glycosaminoglycans (GAGs). GAGs can include carbohydrate polymers attached to extracellular matrix proteins to form one or more proteoglycans. One or more proteoglycans may attract positively charged sodium ions through a net negative charge, which may also attract water molecules, keeping the extracellular matrix hydrated.

An extracellular matrix can be divided into an interstitial matrix and a basement membrane. The interstitial matrix can include one or more gels of polysaccharides or fibrous proteins that may fill interstitial space and may serve or act as a compression buffer against stress placed on an extracellular matrix. The basement membrane can include a sheet-like deposit of extracellular matrix where a variety of epithelial cells rest.

In addition, hyaluronic acid, a polysaccharide, may provide an extracellular matrix with an ability to absorb large quantities of water, which may then allow the extracellular matrix to resist compression with a counteracting swelling force. Moreover, hyaluronic acid may serve as an environmental regulator of cellular behavior during body development, healing processes, inflammation or tumor development.

Structural components or particles of an extracellular matrix may be contained within a liquid transport medium that may be applied to a patient's scalp, and after a period ranging from 24-48 hours, stem cells within a layer of skin on the patent's scalp may be prompted to begin regenerating hair follicles.

The duration of contact between donor sites and extracellular matrix components has been unsatisfactory to date. In particular, structural components of the extracellular matrix are insoluble. In other words, structural components of an extracellular matrix may not be dissolved in solution and injected into injured tissue. Instead, the structural components must be applied directly to a wound or surrounding tissue as a topical treatment that may be absorbed by the skin tissue over time. The necessitated topical delivery mechanism coupled with the length of time required for complete absorption may prevent the treatment from being effective, as any physical contact with the unprotected topical treatment will eliminate its presence. The extracellular matrix components have been imbedded in woven cloths to stabilize their contact with a localized wound site. However, such delivery systems may be relatively expensive and therefore not cost-effective when wounds are small yet numerous and span large surface areas, as seen in follicular unit extractions. As an alternative, structural components of an extracellular matrix may be suspended in a topical gel. Although the topical gel circumvents the economic dilemma of bandages, they may be accidentally or intentionally wiped off the surface of a patient's skin tissue before the active ingredients have taken effect.

Although sterile bandages may be used to cover topical gels to prevent the treatment from encountering physical contact, there remains a possibility that the bandages may become damaged or removed by the patient before the extracellular matrix components have been present on the scalp for a sufficiently long period of time. Furthermore, if the extracellular matrix is delivered in a liquefied solution, there is a possibility that the treatment will be absorbed into the initially dry bandage where it will no longer be in a position to serve its intended function, promoting hair regeneration via stem cell stimulation in follicular unit donor sites.

As discussed herein, a polymeric hydrogel system is provided to allow delivery and/or shielding of topical extracellular matrix components with the specific intention of accelerated wound healing. A biodegradable substance delivery system and method is provided to promote wound healing in a transplantation procedure such as follicular unit extraction and implant sites or locations following a hair transplantation or restoration surgery. A static drug shielding system and method of application is provided for identical purposes.

Efficacious wound treatment may vary drastically based on severity, location, or size of a lesion. While minor scrapes, cuts, and incisions may be mended with topical antibacterial ointment and bandaging, significant injuries and chronic wounds (e.g., diabetic foot ulcers, pressure ulcers, or venous stasis ulcers) may require different treatment approaches. For example, a standard of care for chronic wounds may include off-loading, attentive debridement, maintenance of a moist wound environment, and, if cellulitis is present, systemic antibiotics. Investigations of the pathophysiology of chronic wounds have generally shown that such lesions may display decreased levels of growth factors, topical platelet derived growth factor, tissue growth factor beta (TGF-β), and platelet-derived wound healing factor. Moreover, research shows that treatment methods employing PDGF and TGF-β promote wound healing.

A “growth factor,” as used herein may refer to a substance capable of stimulating cellular growth, proliferation, or cellular differentiation. For example, a growth factor can include a protein or a steroid hormone. A growth factor may act as a signaling molecule between cells. Cytokines and hormones can include examples of growth factors that bind to specific receptors on the surface of their target cells. A growth factor may promote cell differentiation and maturation.

A “platelet-derived growth factor” (PDGF), as used herein, may refer to a growth factor that regulates cell growth or division. In particular, PDGF may play a role in blood vessel formation (angiogenesis) from already-existing blood vessel tissue. In chemical terms, PDGF can include a dimeric glycoprotein composed of two A (-AA) or two B (-BB) chains or a combination of the two (-AB). PDGF can include a potent mitogen for cells of mesenchyme origin, including smooth muscle cells and glial cells. PDGF may be synthesized, stored or released by platelets upon activation.

A “tissue growth factor beta,” or “transforming growth factor beta” (TGF-β), as used herein, may refer to a protein that may control proliferation, cellular differentiation, or other functions in a cell. TGF-β can include a secreted protein that may exist in at least three isoforms such as, e.g., TGF-β1, TGF-β2 and TGF-β3. A TGF-β family is part of a superfamily of proteins known as the transforming growth factor beta superfamily, which can include inhibins, activin, anti-mullerian hormone, bone morphogenetic protein, decapentaplegic or Vg-1.

A “platelet derived wound healing factor” (PDWHF), as used herein, may refer to an extract of activated platelets. A PDWHF may enhance or promote healing of cutaneous ulcers. PDWHF can include an isolated cytokine that may enhance wound healing.

Further research into the addition of extracellular matrix components such as collagens (Types I, III, V, and VII), elastins, glycosaminoglycans (GAGs), and proteoglycans in wound sites shows that these compounds may facilitate wound healing by promoting tissue granulation. Consequently, a number of extracellular matrix component delivery systems have been developed and made available on the market today. These extracellular matrix component delivery systems may be grouped into one of three generic categories: collagen-based, non-collagen extracellular matrix, or biosynthetic composite scaffold. Furthermore, the nature of a delivery system may vary from loose particles to be dispersed in an open wound, to porous sponges or gels that may be placed within the wound, to woven bandages laced with extracellular matrix particles that may be draped over a wound site. Although such systems may work well for isolated extremity or torso sites, such as venous ulcers, these systems may not be economically practical for wounds that span a large surface area, or, in the case of gels, wounds that are positioned on a heavily contacted bodily surface (e.g., a patient's scalp during sleep). Therefore, a cure-on-demand hydrogel coating that may be applied over extracellular matrix treated sites or doped directly with extracellular matrix components may be beneficial to a wound care market.

Thorough and swift wound healing following a hair restoration surgery may be crucial for complete growth of transplanted hair grafts as well as stimulation of stem cells within donor sites to replace extracted follicles. Although extracellular matrix treatments have proven to be efficacious in chronic wound treatment, no delivery system has yet been developed to target wounds spanning a large surface area or to offer aesthetically appealing concealment.

Follicular unit extraction or harvesting may involve removal of individual follicular groups or sections of individual follicular groups. A surgeon or operator may harvest deeply so that all the adipose is removed around the surrounding follicles or one may practice a minimal depth harvest where individual follicles are removed without harm to surrounding adipose. Limiting the depth of the incision may allow a surgeon to extract follicles without adipose. A limited depth incision may allow a physician to remove follicles by plucking them. Plucking may be facilitated via use of a specialized forceps. Plucking hair follicles may leave stem cells intact within the adipose. Under influence of an extracellular matrix, stem cells may generate new hair follicles.

As discussed herein, a photopolymerizable hydrogel system is provided that can include one or more of extracellular matrix collagens, hyaluronic acid, or biotin and which may provide a suitable platform for expediting wound healing in large, irregularly bordered, or load sensitive bodily regions. Such a drug delivery system may initially be viscous or flowing, and upon visible or ultra violet light exposure, the fluid material may transform into an adhesive thin film. Additionally, monomers with biodegradable sections may be selected for this application such that a drug release rate is regulated and may increase over time. Exemption of the biodegradable component would still prove useful, though cross-linked gel would have to be physically removed once the donor sites have absorbed the extracellular matrix components.

A key benefit of such a drug delivery system is the cure-on-demand nature, which may allow an operator to position medication as needed prior to locking a film into place on a patient's skin tissue. Furthermore, application of a viscous or flowing substance may enable maximum coverage of large surface areas with minimal material, thereby minimizing cost. Monomers may readily undergo polymerization to form a biodegradable cross-linked network such that a finished product may release drug contents at a variable rate. Currently used materials may not offer a feature for regulating a rate of release of drug contents. Use of thin films may provide an additional benefit of affording a transparency to aide in concealment of the treatment for cosmetic purposes.

A cure-on-demand, biodegradable drug delivery system as discussed herein may incorporate polyethylene glycol (PEG) (IUPAC name polyethylene oxide or polyoxyethylene) and polycaprolactone (PCL) (IUPAC name (1,7)-polyoxepan-2-one) monomers functionalized with cross-linkable chemical functionalities. In particular, radical mediated chemistry such as the thiol-ene click reaction or the standard (meth)acrylate homopolymerization may be utilized. However, it should be recognized that in either scenario all monomers must contain a minimum of 2 reactive groups, and in the case of the thiol-ene reaction, either the thiol (IUPAC name sulfhydryl) or ene monomer must possess a minimum average functionality of 3 (to promote cross-linking). For example, multiple thiol functionalities may be affixed to a high molecular weight PEG monomer, and terminal acrylate groups may be bound to a linear PEG-PCL block copolymer. However, any unsaturated carbon-carbon double bond or triple bond may be substituted for the acrylate, including but not limited to vinyl, allyl, methacrylate, maleimide, or alkyne. If the thiol component is avoided, the acrylate monomer will homopolymerize in a chain growth reaction process rather than copolymerize with thiols in a step growth reaction process.

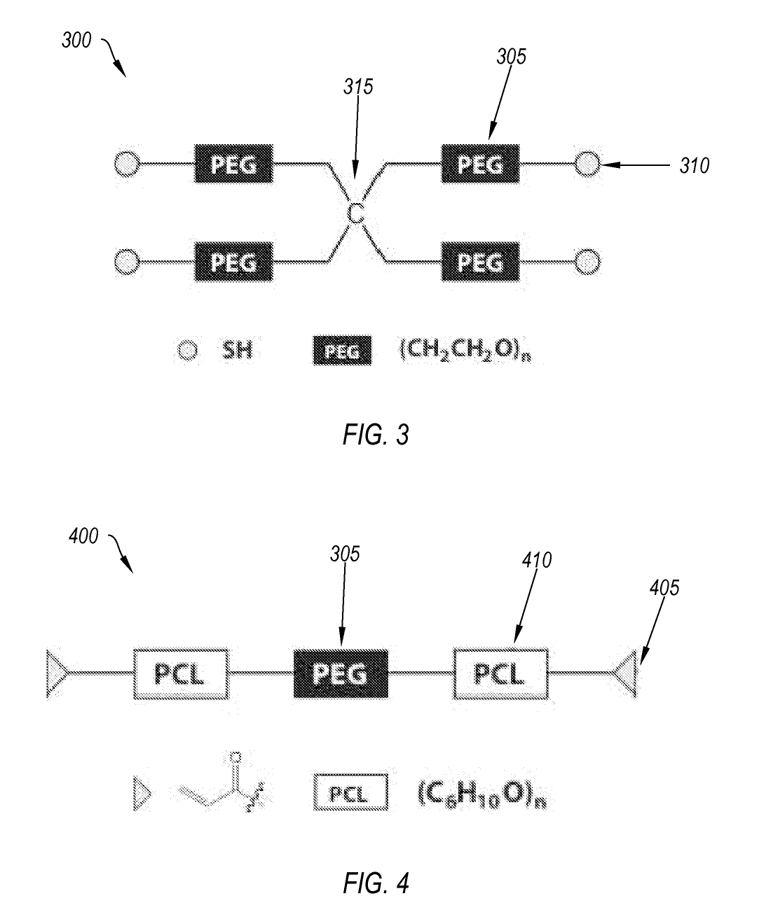

FIG. 3 illustrates an example of a representative multi-arm PEG-thiol monomer 300. As shown, multi-arm PEG thiol monomer 300 can include four separate arms or locations all of which contain a PEG sequence (CH₂CH₂O)ₙ 305. The terminal end of each arm is bound to a thiol 310, which acts as a reactive chain transfer group in the thiol-ene reaction. A thiol 310 molecule has the chemical formula SH. Increasing or decreasing the number of PEG-thiol groups stemming from the central carbon atom 315 may be used to alter the degree of thiol functional ization.

The multi-arm PEG-thiol monomer 300 has four arms, each with a terminal thiol group. Number average molar mass (Mₙ) is determined by the length of the PEG repeat 305 and may be varied to adjust the initial pore size, though the suggested range for the application herein would span 0.5 kDa to 10 kDa. The number of thiols 310 per monomer may range from 2 (a linear configuration) to 8 (a branched configuration). A thiol functionality of 2 will prevent crosslink network formation unless an ene monomer counterpart with functionality >2 is used. Alteration in a degree of thiol functionalization may result in changes to network thermomechanical properties and may be used to tailor the material to a specific application.

FIG. 4 illustrates an example of an acrylate monomer 400 (IUPAC name salt or ester of Prop-2-enoic acid). An acrylate monomer 400 is a chemical compound with a formula CH₂CHCOR. The substituent R may also comprise a polymer such as PEG 305. Acrylates 405 linked to PEG 305 chains may be used as flexible linking molecules to attach proteins to surfaces. A double bond may readily react with a thiol group found on cysteine to form a stable carbon-sulfur bond.

The acrylate monomer may further contain a biodegradable component, such as PCL 410 with the repeat chemical formula (C₆H₁₀O₂)n, though alternative chemical groups may be used (e.g., lactide, glycolide, chitosan, hydroxybutyric acid, or polyphosphazene). FIG. 4 illustrates a representative biodegradable acrylate monomer wherein the polymer chain consists of PCL (A) and PEG (B) blocks in the form of A-B-A. The lengths of A and B may be varied to control the rate of polymer degradation as well as the overall number average molar mass (Mₙ). In some embodiments, the PCL (or similar biodegradable section) may be omitted entirely for a semi-permanent bandage configured to be removed by external force. As used in the specification and the claims, the phrase “configured to” denotes an actual state of configuration that fundamentally ties recited elements to the physical characteristics of the recited structure. As a result, the phrase “configured to” reaches well beyond merely describing functional language or intended use since the phrase actively recites an actual state of configuration.

The number of acrylates per monomer may be adjusted to control the initial pore size and network thermomechanical properties. Functionality greater than 2 would result in a branched or star-shaped monomer with arms extending from a central carbon. The proposed acrylate monomer would have either 2 or 4 acrylates, a molecular weight of approximately 10 kDa, and equal A and B lengths (i.e., the values of n for each block would be the same).

If a solution containing PEG-acrylate or a combination of PEG-acrylate and PEG-thiol monomers is applied without inclusion of a photoinitiator, the solution may be likely to drip off the patient's scalp or be absorbed by bandages. Accordingly, a biocompatible photoinitiator may be formulated in a solution or gel containing either PEG-thiol monomer 300 or PEG-acrylate monomer 400. Application of the appropriate wavelength(s) of light to a biocompatible Type I photoinitiator may cause the photoinitiator to cleave into free radicals. These radicals may then initiate the formation of stable covalent bonds between either sulfur and carbon or carbon and carbon, causing a cross-linked network to be formed. If extracellular matrix components are included in the initial monomer solution, they will become stabilized within the network. Furthermore, when PCL sequences are incorporated into one of the monomer chemical structures, then the cross-linked network will degrade over time. As the network degrades, extracellular matrix will be released. A cross-linked network may provide certain benefits over the gels or woven clothes currently used to apply extracellular matrix particles. Specifically, the cross-linked elastomeric network of this embodiment will be a flexible, thin film that will allow a patient to sleep on a pillow or contact a surface, such as the pillow or a hat, without loss of a substantial portion of the extracellular matrix. Moreover, a biodegradable cross-linked network may provide additional control over the rate of extracellular matrix release. For example, a cross-linked network may degrade over time, and in the process emit extracellular matrix particles into the patient's scalp or skin tissue to promote regeneration of hair follicles via a patient's stem cells.

A biocompatible Type 1 photoinitiator may be formulated within a reaction mixture or solution (approximately 0.01 wt % to 25 wt %) to enable photocrosslinking and instill spatial and temporal control. For example, a lithium acylphosphinate (lithium phenyl-2,4,6-trimethylbenzoylphosphinate) or some other biocompatible species (e.g., riboflavin and a co-initiator such as arginine) may be employed. A system may be utilized under aqueous conditions for multiple purposes, including reduction of final reaction temperature and network stiffness and increase in drug mobility and material flow. An LED dental curing unit may be used as a source of visible light (with some overlap into the UV range) given a proven ability of an LED dental curing unit to induce full conversion of dental composites in short times, emit multiple series of illumination before recharging, and irradiating for pre-set timeframes, as discussed below with respect to FIGS. 8-10.

Therapeutic agents may be formulated within an initial delivery mixture (e.g., approximately 0.1 wt % to 50 wt % in total). Therapeutic agents can include biotin, hyaluronic acid, or extracellular matrix components (collagens, GAGs, or proteoglycans, to name just a few examples) in any combination and concentration. Structural extracellular matrix components may be porcine or bovine derivatives or biomimetic amino acid sequences. Delivery of therapeutic agents may follow a hair restoration surgery such that drugs may have a maximum potential to regenerate hair. However, a cure-on-demand delivery system may potentially be applied to any external wound site.

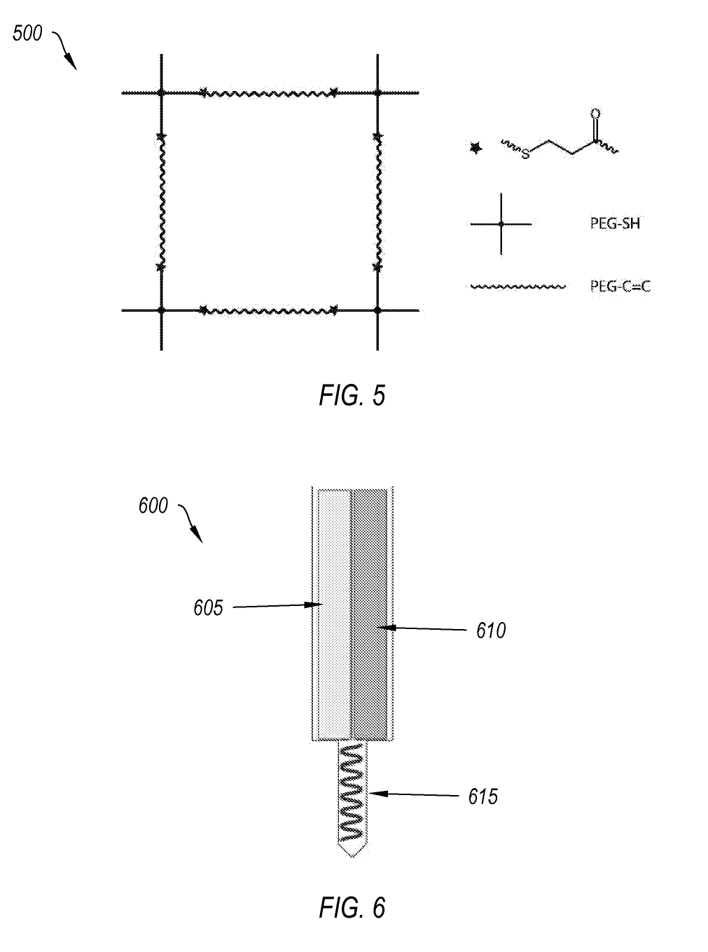

FIG. 5 illustrates an example of a cross-linked network 500. Cross-linked network 500 may be formed via a polymerization process from multi-arm PEG-thiol monomer 300 (FIG. 3) and linear acrylate monomer 400 (FIG. 4). Similarly, a cross-linked network with lower pore size regularity may be formed by photopolymerization of the acrylate monomer alone. As discussed above, a water-soluble, biocompatible photoinitiator may be added to either or both of the aqueous monomer solutions. If a light of a certain wavelength (e.g., approximately 365 nm to 460 nm) is applied to a formulated solution, the photoinitiator will be cleaved into one or more radicals capable of initiating polymerization. In the thiol-ene reaction, initiation will lead to alternating propagation and chain transfer events that enable covalent sulfur-carbon bonds to be formed. When sufficient covalent bonds have been made, the aqueous solution will transition into a hydrogel. The mechanical strength of the network will increase directly with functional group conversion, reaching a maximum when all reactive groups have formed covalent linkages. By forming such bonds, a cross-linked network may be formed on a patient's scalp or skin tissue.

In the event that the thiol component is eliminated, the reaction will proceed solely through propagation events following initiation. Carbon-carbon bonds between acrylate groups in solution will be formed, and a cross-linked network will result. Network strength will again correlate directly with functional group conversion.

The cross-linked network 500 can include high concentrations of PEG and PCL or solely PEG. The inclusion of PCL within one of the monomer structures will instill a biodegradable feature to the cross-linked network. The overall concentration of PEG and PCL will be dependent on the respective sequence lengths within the monomers. For example, should the PEG-thiol (FIG. 3) and PEG-acrylate (FIG. 4) be copolymerized, the network concentration of PEG will be in excess of the PCL concentration since the PEG-thiol does not contain PCL repeats. Similarly, should the PEG-acrylate monomer be polymerized alone, the concentration of PCL will be in excess of PEG owing to the PCL-PEG-PCL monomer structure. Should a variant of the PEG-acrylate monomer be employed wherein the n repeat value of PCL is zero, the network concentration of PCL will also be zero, and the network will not degrade in an appreciable timeframe.

FIG. 6 illustrates an example of a syringe 600 for providing one or more solutions containing one or more monomers to a patient's scalp or skin tissue. As shown, syringe 600 may include a first barrel 605 and a second barrel 610, although it should be appreciated that a syringe 600 may be utilized that has only one barrel or more than two barrels. A multiple barrel syringe delivery method may replace or supplement a photopolymerizable delivery method. A solution containing a PEG-thiol monomer, such as that shown in FIG. 3, may be contained within first barrel 605. A solution containing acrylate monomer, such as that shown in FIG. 4, may be contained within second barrel 610. It should be appreciated that different monomers or other solutions may be contained within first barrel 605 or second barrel 610. Either or both of first barrel 605 or second barrel 610 can include a biocompatible photoinitiator, as discussed above. If the photoinitiator is omitted, the acrylate monomer within second barrel 610 must be replaced with a maleimide monomer. Alternatively, the acrylate monomer within second barrel 610 could be replaced with a primary amine if the thiol monomer within the first barrel 605 is replaced with an activated ester, such as succinimidyl ester. In both instances, an alternative to the radical-mediated thiol-ene reaction mechanism would be undertaken that requires monomers to be isolated from one another prior to application. The thiol-maleimide reaction, for example, proceeds rapidly at pH 6.5 to 7.5 to form a stable thioether bond. Likewise, the conjugation of succinimidyl esters with primary amines occurs spontaneously at pH 7.0 to 8.5, giving rise to a stable amide bonds.

It should be appreciated that a monomer solution contained within first barrel 605 may be kept separate from a monomer solution contained within second barrel 610 to avoid initiating a polymerization process prior to applying monomer solutions to a patient's scalp or skin tissue.

A solution contained within first barrel 605 or second barrel 610 can include hyaluronic acid, biotin, or extracellular matrix particles as a therapeutic agent. Hyaluronic acid is an instrumental factor in establishing epidermal homeostasis and repair in wounded tissue, while biotin is purported to strengthen existing hair.

If first barrel 605 contains a multi-arm PEG-thiol monomer it should be appreciated that the PEG-thiol monomer may be contained within a solution. Such a solution may be purely aqueous and possess a low viscosity. Alternatively, the PEG-thiol monomer can include a mixture of water and gel such that the mixture possesses a higher viscosity. I.e., the precise viscosity may be adjusted by the ratio of water to gel in the PEG-thiol mixture. The chosen viscosity may depend, for example, on the particular application for use or on the shape of a patient's scalp or skin tissue. It should be appreciated that if a patient's scalp has numerous or relatively deep wounds, a solution having a relatively low viscosity may readily flow down into a relatively deep wound, whereas a solution having a relatively high viscosity may not readily flow down into a relatively deep wound and may instead be manually spread across or into a wound via a surgical operator's hand or via use of a surgical instrument.

Certain chemicals or materials may be added or applied to a monomer solution to alter the viscosity of the monomer solution. For example, water may be added to a monomer solution to decrease viscosity. Additionally or alternatively, an elevated level of hyaluronic acid gel may be added to a monomer solution to increase viscosity.

It should be appreciated that second barrel 610 can include a solution having a selected or chosen viscosity based on a particular application. For example, both first barrel 605 and second barrel 610 can include the same or similar solutions having similar viscosities. In certain embodiments, however, it may be advantageous to use different solutions having different viscosities in first barrel 605 and second barrel 610.

A surgical operator may utilize syringe 600 to expel solutions containing thiol and acrylate monomers simultaneously from the first barrel 605 and the second barrel 610 to be mixed through a static mixer tip 615. In some embodiments, however, a solution from a first barrel 605 may be applied to a wound at a time different from that of the solution in the second barrel 610.

One of skill in that art will appreciate that a device other than a syringe 600 may be utilized to apply a monomer solution to an area of a patient's scalp or skin tissue. For example, a spray nozzle may be utilized to spray a monomer solution onto an area of a patient's scalp. Additionally or alternatively, a monomer solution may be poured or dripped onto an area of a patient's scalp from some apparatus or device other than a syringe 600.

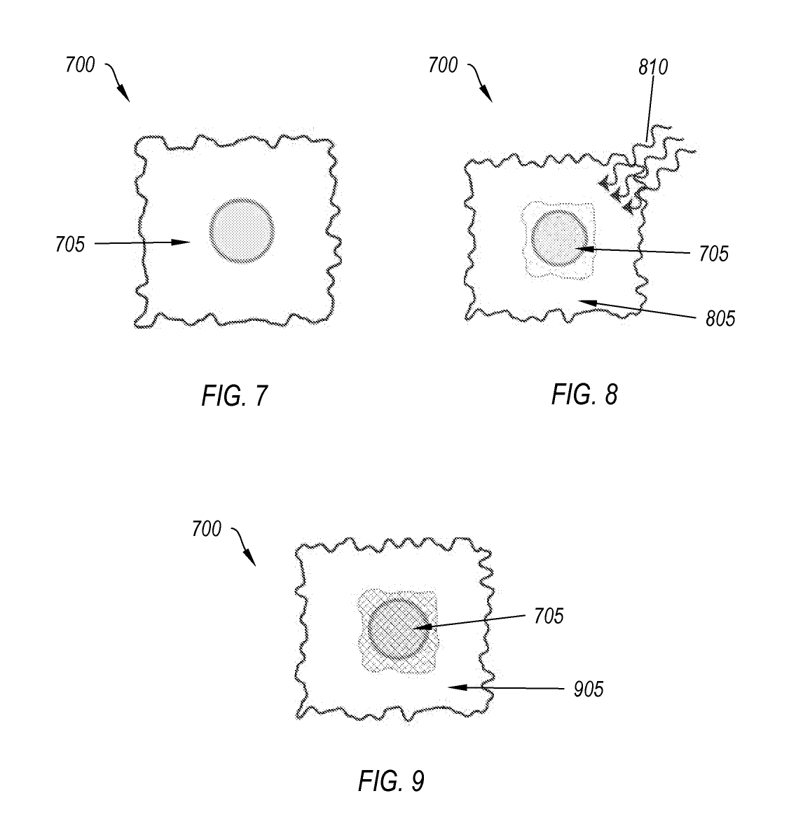

FIG. 7 illustrates an example of an area of a patent's hair growing tissue 700 having a wound 705. For example, wound 705 may be created during a hair follicle harvesting or extraction process. Extracellular matrix components may be applied to wound 705 to instruct stem cells within a layer of skin in patient's scalp to regenerate removed hair follicles, as discussed above.

FIG. 8 illustrates an example of an area of a patent's hair growing tissue 700 having a wound 705 with monomer solutions 805 applied. Monomer solutions 805 may be applied to a portion of a patient's hair growing tissue 700, such as wound 705, via use of a syringe, such as syringe 600 shown in FIG. 6. After applying monomer solutions 805 to wound 705, the monomer solutions 805 may be irradiated with one or more wavelengths of light 810 from a light source. Energy from one or more wavelengths of light 810 may be absorbed by biocompatible photoinitiators contained within monomer solutions 805 to initiate a polymerization process and create a cross-linked network, e.g., as shown in FIG. 5. A particular wavelength of light may be selected based on the absorption profile of a particular type of biocompatible photoinitiator contained in monomer solution(s) 805. For example, LED units having a wavelength between approximately 370 and 460 nm may be utilized to irradiate a water-soluble photoinitiator, lithium acylphosphinate. On the other hand, if a certain biocompatible photoinitiator system such as riboflavin with a suitable co-initiator such as L-arginine is utilized, light beams having a wavelength of approximately 340-415 nm may be utilized to irradiate a monomer solution 805. In certain embodiments, ultraviolet light may be preferable to irradiate a particular monomer solution, and in others visible light may be preferable.

The length of time for irradiating a monomer solution may be dependent upon the concentration of monomer in solution. For example, if a monomer solution 805 contains very few monomer molecules relative to water molecules or some other liquid, a polymerization process may occur more slowly than may occur if a monomer solution 805 contains a large number of monomer molecules. Similarly, the intensity level of light output from the light source 810, may affect the length of time for performing a polymerization process.

FIG. 9 illustrates an area of a patent's hair growing tissue 700 having a wound 705 with a cross-linked network 905 disposed on the wound 705. As shown, a cross-linked network 905 may be disposed across a top surface of wound 705. A cross-linked network 905 may be created by irradiating a monomer solution with a light source as discussed above with respect to FIG. 8.

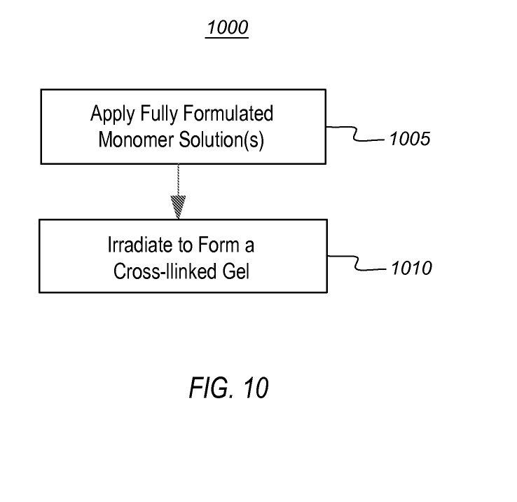

FIG. 10 illustrates a process 1000 for generating a cross-linked network. The order of blocks 1005-1010 comprises an example order. Claimed subject matter is not limited in scope to illustrative or example embodiments. Therefore, embodiments in accordance with claimed subject matter may include all of, less than, or more than blocks 1005-1010. At operation 1005, a monomer solution, series of solutions, or combination of solutions may be applied to a tissue area of a patient. At least one monomer solution can include a therapeutic agent. At operation 1010, a polymerization process may be initiated in a monomer solution or between two monomers mixed in a solution to create a hydrogel network to deliver or confine a therapeutic agent to a tissue area.

Light may be used to initiate a polymerization resulting in cross-linked network formation. A cross-linked network can include a hydrogel. A photoinitiator species can include lithium acylphosphinate. A therapeutic agent can include components of an extracellular matrix, biotin, hyaluronic acid, or some variation thereof.

A PCL block length may be selected to adjust a rate of biofilm degradation. A degree of reactive species functionalization (e.g., thiol or ene) may be selected to adjust a rate of biofilm degradation. A molecular weight of cross-linking monomers may be selected to adjust a rate of release of a therapeutic agent. In accordance with an embodiment, a pre-polymerized formulation may be delivered locally.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for creating a polymeric hydrogel for accelerating wound healing, the method comprising:
applying at least one monomer solution to a tissue area of a patient, wherein the monomer solution includes:
a monomer; and
a therapeutic agent; and
initiating a polymerization process in the applied monomer solution to create a cross-linked network to deliver the therapeutic agent to the tissue area.

2. The method of claim 1 wherein the monomer includes at least one polyethylene glycol block.

3. The method of claim 1 wherein the monomer includes at least one polycaprolactone block.

4. The method of claim 1 wherein the monomer solution includes a second monomer.

5. The method of claim 4 wherein:
the monomer includes a terminal vinyl; and
the second monomer includes a terminal thiol.

6. The method of claim 4 wherein the block length of the PCL is selected to adjust a rate of biofilm degradation.

7. The method of claim 1 wherein the reactive moieties on the monomer are configured to ensure that a cross-linked network is formed.

8. The method of claim 1 wherein the monomer includes a section configured to breakdown over a predetermined period of time to regulate release of the therapeutic agent.

9. The method of claim 1 wherein the skin tissue comprises at least a portion of a scalp.

10. The method of claim 1 wherein the skin tissue includes an area previously subjected to a hair follicular extraction procedure.

11. A system for creating a polymeric hydrogel for accelerating wound healing, the system comprising:
a first apparatus configured to apply a monomer solution to a tissue area of a patient, wherein the monomer solution includes:
a monomer; and
a therapeutic agent; and
a second apparatus configured to initiate a polymerization process in the applied monomer solution to create a cross-linked network to deliver the therapeutic agent to the tissue area.

12. The system of claim 11 where the first apparatus includes a syringe.

13. The system of claim 11 wherein the first apparatus includes a spray nozzle.

14. The system of claim 11 wherein the monomer solution includes a photoinitiator.

15. The system of claim 14 wherein the second apparatus is configured to irradiate the photoinitiator with one or more wavelengths of light.

16. A system for creating a polymeric hydrogel for accelerating wound healing, the system comprising:
a double barreled syringe configured to apply a monomer solution to a tissue area of a patient, wherein the monomer solution includes:
a first monomer in a first barrel of the double barreled syringe;
a second monomer in a second barrel of the double barreled syringe; and
a therapeutic agent in the first barrel; and
an apparatus configured to initiate a polymerization process in the applied monomer solution to create a cross-linked network to deliver the therapeutic agent to the tissue area.

17. The system of claim 16 wherein the therapeutic agent includes at least one of:
biotin; or
hyaluronic acid.

18. The system of claim 16 wherein the double barreled syringe includes a mixer tip configured to mix the first monomer and the second monomer.

19. The system of claim 16 wherein the therapeutic agent includes components of an extracellular matrix configured to stimulate one or more stem cells.

20. The system of claim 19 wherein at least partially in response to stimulating the one or more stem cells, hair follicles are regenerated.
